(19) United States
(12) Patent Application Publication
(10) Pub. No.: US 2010/0274101 A1
(43) Pub. Date: Oct. 28, 2010

(54) WIRELESS MONITORING BIO-DIAGNOSIS SYSTEM

(75) Inventors: Chii-Wann Lin, Taipei (TW); Kuang-Chong Wu, Taipei (TW); Chih-Kung Lee, Taipei (TW); Shi-Ming Lin, Taipei (TW); Shih-Yuan Lee, Taipei (TW); Fu-Shan Jaw, Taipei (TW); Chern-Lin Chen, Taipei (TW); U Lei, Taipei (TW); Long-Sun Huang, Taipei (TW); Shey-Shi Lu, Taipei (TW); Phone Lin, Taipei (TW); Jia-Yush Yen, Taipei (TW); Yao-Joe Yang, Taipei (TW); Lung-Jieh Yang, Taipe (TW); Wen-Pin Shih, Taipei (TW); Nan-Fu Chiu, Taipei (TW)

Correspondence Address: SCHMEISER, OLSEN & WATTS, 22 CENTURY HILL DRIVE, SUITE 302, LATHAM, NY, 12110, US

(73) Assignee: NATIONAL TAIWAN UNIVERSITY, TAIPEI (TW)

(21) Appl. No.: 12/556,675

(22) Filed: Sep. 10, 2009

(30) Foreign Application Priority Data
Apr. 24, 2009 (TW) 98113628

(51) Int. Cl. A61B 5/00

(52) U.S. Cl. 600/301

(57) ABSTRACT

A MEMS wireless monitoring bio-diagnosis system includes an implantable biosensor system chip, a surface transmitter and an external monitor center. The implantable biosensor system chip contains a biosensor for a cardio-vascular indicator and a wireless transmitter to deliver detected bio-signal data. With the MEMS wireless monitoring bio-diagnosis system, the bio-signal data can be monitored effectively and transmitted to a remote medical unit.

### FIELD OF THE INVENTION

The present invention relates to a wireless monitoring bio-diagnosis system using a wireless transmission technology for physiological monitoring, in particular to a wireless monitoring bio-diagnosis system using an implantable biosensor system chip together with a wireless transmission technology for the physiological monitoring.

### BACKGROUND OF THE INVENTION

Nowadays, chronicle diseases including diabetes and cardiovascular disease are very common, and thus diabetes, cerebrovascular disease, heart disease and hypertension are always listed as the top 10 causes of death in Taiwan. Since blood sugar concentration, blood pressure, blood lipid and other physiological signals of these chronicle diseases must be controlled and monitored on a long-term basis. For example, diabetic patients have to monitor their blood sugar concentration everyday in order to effectively reduce the risk of heart diseases complicated with hypertension and myocardial infarction for diabetic patients. Therefore, it is an important subject for researchers to develop biomedical monitoring systems of this sort and obtain a patient's physiological signals effectively and conveniently and transmits the physiological signals to a medical center as a basis for diagnosing a patient's physical conditions.

In a conventional biomedical monitoring system, a physiological signal monitoring device is connected externally to a patient's body for capturing physiological signals of a monitoring patient periodically, and the patient's physiological signals are monitored by an external instrument to obtain the patient's data, and the data are transmitted to a medical center or a monitoring center as a reference basis for analyzing the patient's physical conditions. However, conventional instruments of this sort (e.g. glucose sensors) are non-intrusive glucose sensors situated outside a patient's body, and thus their accuracy and reliability are not as good. Therefore, a biomedical monitoring system having a subcutaneous implant sensor with a low level of intrusiveness becomes a development trend of the biomedical monitoring systems.

In a subcutaneous implant sensor of a conventional biomedical monitoring system, the properties of the subcutaneous implant sensor including a bio-compatibility, an interference resistibility, a structural durability and a monitoring effect cannot be enhanced due to limitations of its design. Furthermore, the subcutaneous implant sensor transmits the monitored physiological signals to an external surface transmitter by different transmission methods. The conventional subcutaneous implant sensors are unable to achieve the effect of timely monitoring the required physiological signals at a remote end, and the diagnosis of the patient's conditions may be delayed.

### SUMMARY OF THE INVENTION

Therefore, it is a primary objective of the present invention to provide a wireless monitoring bio-diagnosis system to overcome the shortcomings including a poor stability of the sensors and an inconvenient signal transmission in a conventional biomedical monitoring system. The wireless monitoring bio-diagnosis system of the present invention is provided for achieving a remote medical monitoring effect to monitor and control a patient's physiological signals anytime. A wireless network module is used to collect and transmit data of the physiological signals to an external monitor center for further analyses. With the wireless monitoring bio-diagnosis system of the present invention, we can overcome the shortcomings of a conventional wireless monitoring bio-diagnosis system effectively, and thus the invention is an effective, convenient and novel technology.

To achieve the foregoing objective, a preferred embodiment of the present invention provides a wireless monitoring bio-diagnosis system comprising one or more implantable biosensor system chips, a surface transmitter and an external monitor center, wherein the implantable biosensor system chip is connected to the surface transmitter via a wireless network and further connected to an external monitor center via an external network. The implantable biosensor system chip comprises a biocompatible package, a biosensor and a wireless transmitter, wherein the biosensor and the wireless transmitter are wrapped inside the biocompatible package. The biocompatible package can be made of polyurethane (PU), polyethylene (PE), polymethylmethacrylate (PMMA), polyester (PE), poly tetra fluoro ethylene (PTFE), polydimethylsiloxane, poly tetramethylene succinate (PTMS) or any other polymer with a good biocompatibility. The biosensor includes a dielectrophoreis electrode for driving fluids and separating blood, an electrochemical detection electrode, and an inlet and an outlet disposed on both ends of the biosensor respectively. In the aforementioned implantable biosensor system chip, the wireless transmitter includes a RF power device. In an implantable biosensor system chip adopted by the wireless monitoring bio-diagnosis system of the present invention, the chip is provided for monitoring heartbeat, blood sugar, enzyme concentration, protein concentration or other physiological signals. Preferably, lactate dehydrogenase, glucose oxidase or C-reactive protein, S-100 protein indicating a cardiovascular disease is monitored.

### BRIEF DESCRIPTION OF THE DRAWINGS

FIG. 1 is a schematic view of a wireless monitoring bio-diagnosis system of the present invention;

FIG. 2 is an integrated system block diagram of a wireless monitoring bio-diagnosis system of the present invention;

FIG. 3 is a system block diagram of a biomedical wireless body area network system of the present invention;

FIG. 4 is an overall schematic view of an implantable biosensor system chip adopted by a wireless monitoring bio-diagnosis system of the present invention;

FIG. 5A illustrates a structure of an implantable biosensor system chip biosensor in a wireless monitoring bio-diagnosis system of the present invention;

FIG. 5B is a schematic view of a channel electrode of a biosensor of an implantable biosensor system chip in a wireless monitoring bio-diagnosis system of the present invention;

FIG. 6A illustrates a protein antibody/antigen sensing mechanism of an implantable biosensor system chip biosensor in a wireless monitoring bio-diagnosis system of the present invention;

FIG. 6B illustrates a glucose sensing mechanism of an implantable biosensor system chip biosensor in a wireless monitoring bio-diagnosis system of the present invention;

FIG. 6C illustrates a lactate sensing mechanism of an implantable biosensor system chip biosensor in a wireless monitoring bio-diagnosis system of the present invention; and

FIG. 7 illustrates a method of manufacturing an implantable biosensor system chip in a wireless monitoring bio-diagnosis system of the present invention.

### DESCRIPTION OF THE PREFERRED EMBODIMENTS

Other features and advantages of the present invention will become apparent in the following detailed description of the preferred embodiments with reference to the accompanying drawings.

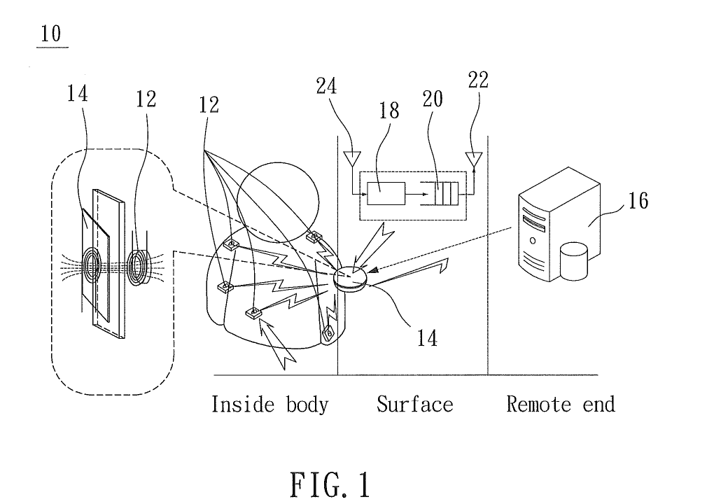

With reference to FIG. 1 for a wireless monitoring bio-diagnosis system of the present invention, the wireless monitoring bio-diagnosis system 10 comprises one or more implantable biosensor system chips 12, a surface transmitter 14 and an external monitor center 16, wherein the implantable biosensor system chip 12 is connected to the surface transmitter 14 via a wireless network and further connected to an external monitor center via the Internet. The surface transmitter 14 can be worn securely on a surface of the examinee's body or a handheld surface transmitter 14 of an implantable biosensor system chip 12 is placed near the examinee's body when it is necessary to obtain data of physiological signals. In FIG. 1, the examinee's body includes a plurality of implantable biosensor system chips 12, and the plurality of implantable biosensor system chips 12 are biosensors provided for measuring various physiological signals such as heartbeat, blood sugar level, enzyme concentration and protein concentration, and transmitting one or more of the obtained physiological signals to a wireless transmitter of the surface transmitter 14 via a wireless transmission. After the surface transmitter 14 receives the physiological signals transmitted from the plurality of implantable biosensor system chips 12 as shown in FIG. 1, the surface transmitter 14 buffers the data into a memory 18. After a sorter 20 sorts the data in an appropriate sequence, a wireless transmitter 22 transmits the data of the physiological signals to an external monitor center 16. The external monitor center 16 can be an external server, a workstation or any other similar device connected to the surface transmitter 14 via a cable or a wireless transmission of the Internet for obtaining data or issuing an instruction. After the external monitor center 16 obtains the data of the physiological signals from the surface transmitter 14, a database installed in the external monitor center 16 is used for comparing and analyzing whether or not the obtained physiological signals are abnormal. In the meantime, the obtained physiological signals are stored in the external monitor center 16 to produce a database of physiological signals. If the obtained physiological signals are compared and determined as abnormal, then the external monitor center will issue an instruction to the surface transmitter 14 to request another measurement of the physiological signals again or additionally monitor other necessary physiological signals. Since the external monitor center 16 is managed by medical professionals, the abnormal physiological signals can be outputted and analyzed and provided for the medical professionals to interpret and take necessary actions. In the meantime, the medical professionals can control the surface transmitter 14 through the external monitor center 16 to command the plurality of implantable biosensor system chips 12 to capture different physiological signals.

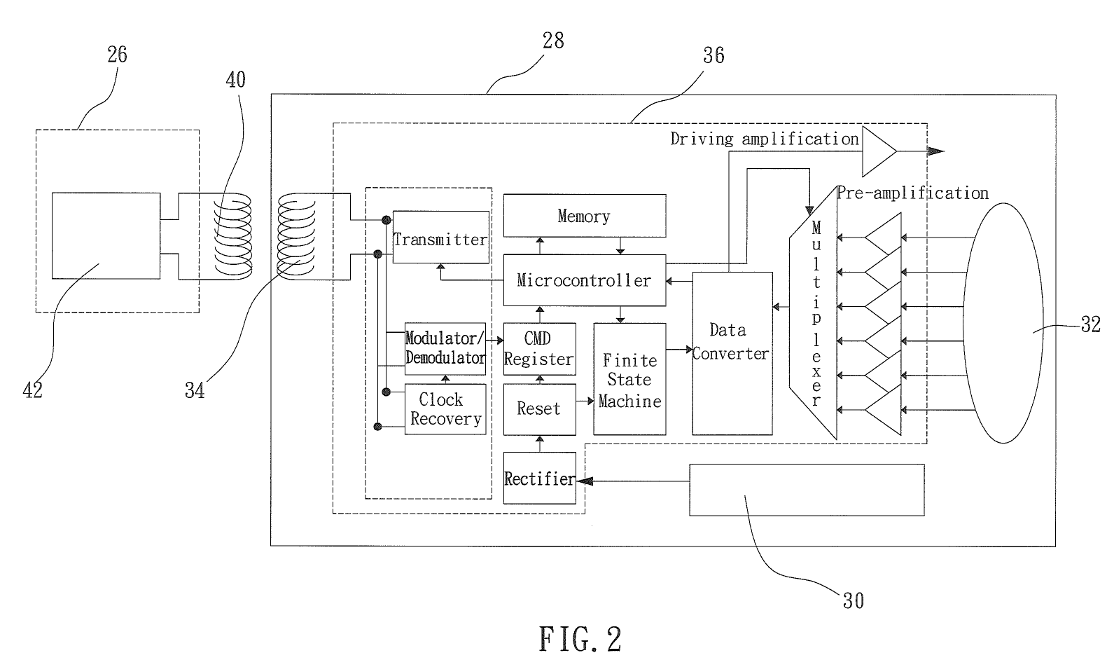

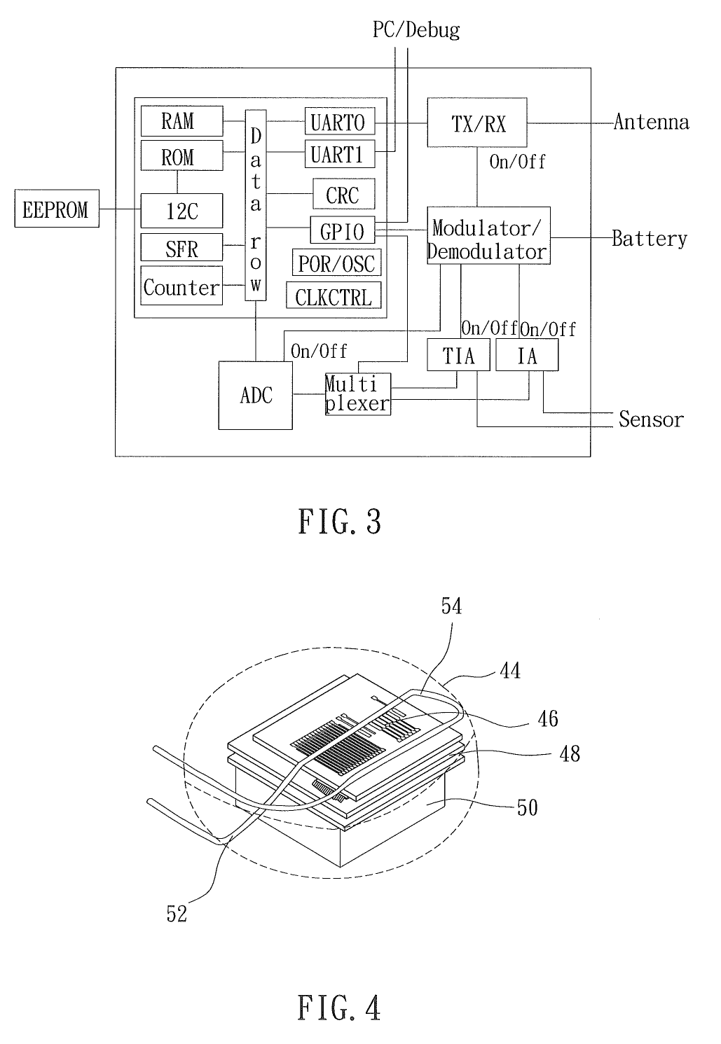

With reference to FIG. 2 for an integrated system block diagram of a wireless monitoring bio-diagnosis system of the present invention, an integrated system architecture of the present invention of a wireless monitoring bio-diagnosis system includes a surface transmitter 26 and a biosensor system chip 28 implanted into a human body. The surface transmitter 26 includes a control portion 42 for controlling and buffering data and instructions and an antenna 40 for sensing an implant biosensor system chip 28. The implant biosensor system chip 28 includes a biosensor 32 for obtaining physiological signals, a wireless transmitter 36 for processing the collected data of the physiological signals and transmitting the data to the surface transmitter 26, an antenna 34 for transmitting the data to the surface transmitter 26, and a battery 30 for supplying electric power to the biosensor 32, the wireless transmitter 36, and the antenna 34. The control portion 42 of the surface transmitter 26 as shown in FIG. 1 buffers the data into a memory of the control portion. After a sorter sorts the data into an appropriate sequence, the wireless transmitter transmits the data of the physiological signals to an external monitor center, or the external monitor center transmits an instruction to each implant biosensor system chip 28 through an antenna 40. The implant biosensor system chip 28 has a structure as shown in FIG. 2, wherein the detailed structure and the operating principle of the biosensor 32 will be described later. In a sensing process of the implant biosensor system chip 28, the biosensor 32 transmits the obtained data of the physiological signals to a multiplexer of the wireless transmitter 36, and then transmits the data to a transmitter connected to the antenna 34 after going through the internal data conversion and buffering procedures. The data are transmitted to the surface transmitter 26 for following transmissions. If an instruction transmitted from the external monitor center is received, the instruction will be transmitted from the surface transmitter 26 to an internal clock recovery and modulation/demodulation reader through the antenna 34 and processed by a microcontroller, and the received instruction will be transmitted to the biosensor 32 to execute the instruction. The battery 30 in the implant biosensor system chip 28 can be a wireless chargeable battery for supplying current to the biosensor 32, the wireless transmitter 36 and the antenna 34 through a rectifier. The implant biosensor system chip 28 comprises software and hardware, wherein the software includes a network channel allocation of the implantable biosensor system chip, an output power protocol, a path protocol applicable for inside-body networks, a network-topology decision making system and an establishment of a physical condition database for monitoring bio information and providing medical references. The hardware is similar to the aforementioned biosensors 32 for monitoring different types of physiological parameters and the wireless transmitter 36 and the antenna 34 provided for transmitting and receiving signals from a micro wireless network. The implantable biosensor system chip 28 of the present invention is a bio medical wireless system on a chip (BMW-SoC), whose detailed architecture is shown in FIG. 3. In the system on a chip of the present invention, a voltage and current amplification circuit (IA/TIA) of an integrated circuit is integrated into a system chip (including MCU/ADC/RF) for amplifying various voltage or current type sensing signals detected by the electrochemical detection electrode. In the meantime, the issue of having insufficient power for the implantable biosensor system chip is taken into consideration, and a power management circuit is also integrated into the system chip to reduce the consuming power of the chip. With the aforementioned integration, a chip similar to the bio medical wireless-system on chip (BMW-SoC) adopted by the wireless biomedical monitoring system of the present invention can be achieved. The system on a chip can be a wireless monitoring bio-diagnosis system of the present invention as shown in FIG. 1, and the implant biosensor system chip can be used as a platform provided for the surface transmitter and the external monitor center to monitor an examinee's physical conditions.

With reference to FIG. 4 for an overall schematic view of an implantable biosensor system chip adopted by a wireless monitoring bio-diagnosis system of the present invention, the implantable biosensor system chip comprises a biocompatible package 44, a biosensor 46, a wireless transmitter 48, a battery 50, an antenna (not shown in the figure), an inlet 52 and an outlet 54. The implantable biosensor system chip is implanted into an examinee's body by adopting a subcutaneous implantation method, and detected physiological signals corresponding to reactions in the examinee's body are detected, and data of various physiological signals and identity information of the implanter are read as various types of wireless transmission signals as shown in FIG. 4, and blood is passed through the inlet 52 to the biosensor 46 and then flowed out from the outlet 54, and an electrode of the biosensor 46 is used for collecting desired data of the physiological signals detected by the electrode. The collected data of the physiological signals are transmitted into the wireless transmitter 48, whose detailed structure is illustrated in details in FIGS. 2 and 3 and provided for transmitting internal data of the physiological signals to the outside and returning external instructions. If necessary, the wireless transmitter 48 stores data for confirming different examinee's identities, such that when the external monitor center is connected via a wireless network, the external monitor center is linked directly to a medical record database of the implanter. In the meantime, the battery 50 installed at the bottom of the implantable biosensor system chip supplies electric power required for the operations of the biosensor 46, the wireless transmitter 48 and the antenna (not shown in the figure). The antenna can be a coil installed at the bottom of the battery or any other appropriate antenna, such that the wireless transmission system can transmit electric power to the coil of the implantable biosensor system chip and store the electric power into the battery 50. The implantable biosensor system chip adopted in the monitoring bio-diagnosis system of the present invention wireless is used for confirming an examinee's identity to simplify the medical diagnosis procedure effectively. In the chip implantation process, the biocompatible package 44 is capable of preventing injuries caused by the implantation, and the biocompatible package 44 can be made of polyurethane (PU), polyethylene (PE), polymethylmethacrylate (PMMA), polyester (PE), poly tetra fluoro ethylene (PTFE), polydimethylsiloxane, poly tetramethylene succinate (PTMS) or polymethylmethacrylate (PMMA). The implantable biosensor system chip must use a low current/voltage driving system for the implantation. In the meantime, the technical characteristics of power-saving or low power consumption technologies are required. Therefore, the wireless monitoring bio-diagnosis system of the present invention can supply sufficient electric power to the implantable biosensor system chip, and the electric power is transmitted and supplied through the wireless transmission system to the coil or the antenna of the chip in the examinee's body.

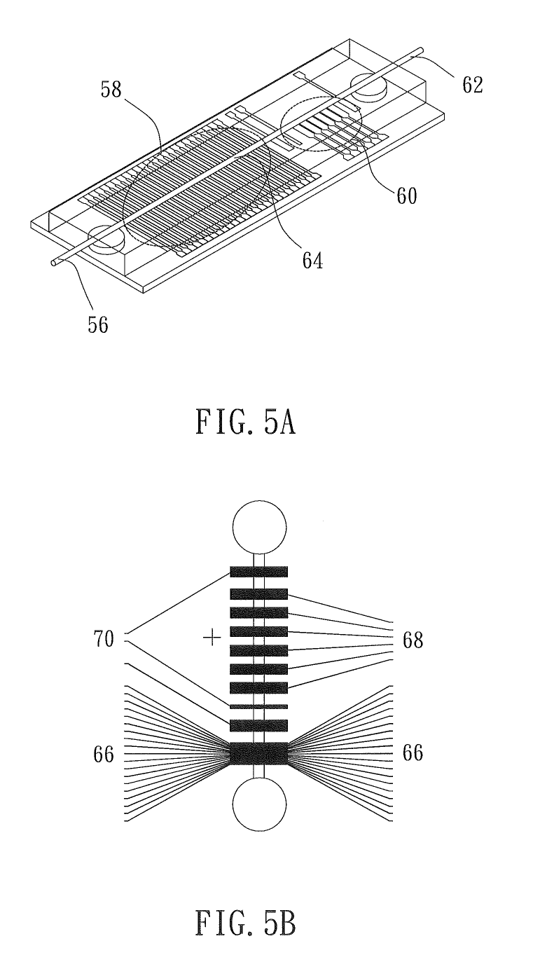

With reference to FIG. 5A for a structure of a biosensor of an implantable biosensor system chip in a wireless monitoring bio-diagnosis system of the present invention, the biosensor comprises a micropump 58, an electrochemical detection electrode 60, an inlet 56, an outlet 62 and a channel 64. The biosensor in the channel 64 detects a change of concentration of sugar, enzyme or protein such as glucose, cholesterol enzyme, lactate dehydrogenase, glucose oxidase C-reactive protein or S-100 protein by using an electrochemical method. The channel 64 includes an inlet 56 and an outlet 64 disposed at a front end of the channel 64 for circulating blood, and an electrochemical detection electrode 60 disposed at a rear end of the channel 64 and made of platinum or another metal for measuring different physiological signals such as breathing, heartbeat, sugar concentration, enzyme concentration or protein concentration, etc. A micropump 58 is produced at the front end of the channel 64 for driving fluids and separating blood, and the micropump 58 is a dielectrophoreis electrode 66 such as the channel electrode as shown in FIG. 5B, and the channel includes three types of electrodes, respectively: a chemical electrode 68, a dielectrophoreis (DEP) electrode 66 and a physical electrode 70. The chemical electrode includes two sets of electrodes and shares a silver electrode, a platinum electrode or another metal electrode to measure glucose or lactic acid, and another set of electrodes is used for an electrochemical measurement of a current value to obtain a concentration change value of other testing physiological signals such as those for the protein and enzyme. The dielectrophoreis electrode 66 in the micropump 58 integrates an application of a traveling wave technology. The micropump 58 is used for driving fluids and separating blood concurrently. In the micropump 58, blood plasma and other testing substances in the blood can be separated by using a traveling wave technology and an electrochemical action, such that the electrochemical detection electrode 60 at the rear end can obtain a more accurate detection result. If the blood is passed through the electrochemical detection electrode 60 and the required data of the physiological signals are obtained, then the blood will return to the examinee's body through the outlet 62. Two major reaction mechanisms including a charge producing oxidase catalytic reaction and an affinity interaction are adopted and provided for the biosensor to measure parameters, but each measured parameter in the whole biosensor is integrated into a single system. After a measurement takes place each time, users can reset to continue taking the next measurement without the need of taking out the system again.

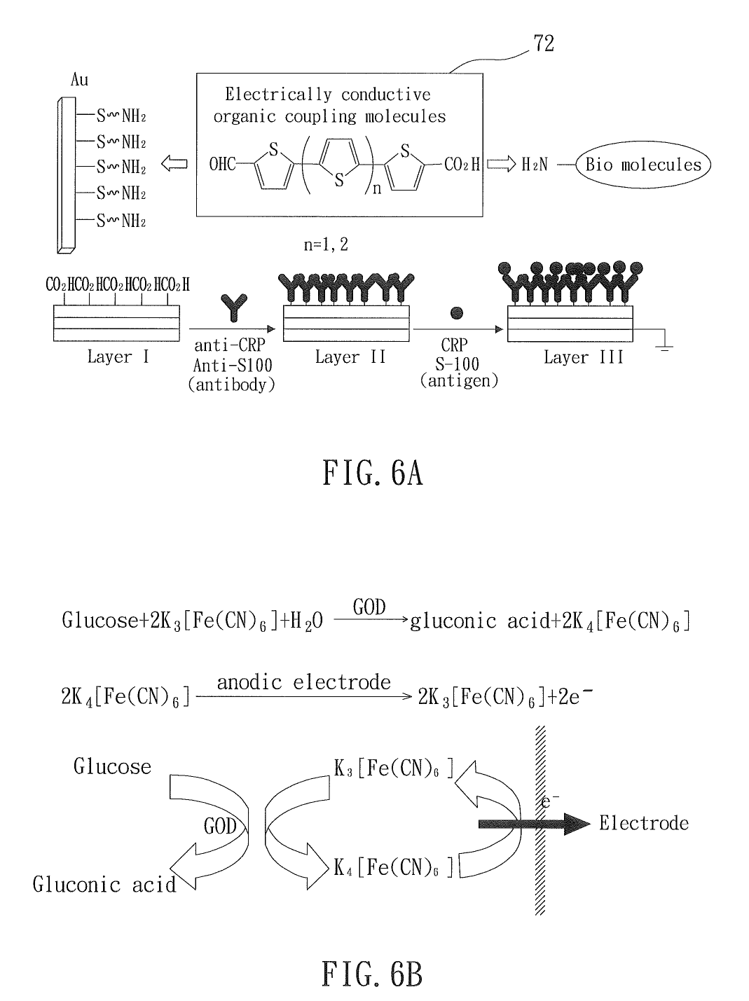

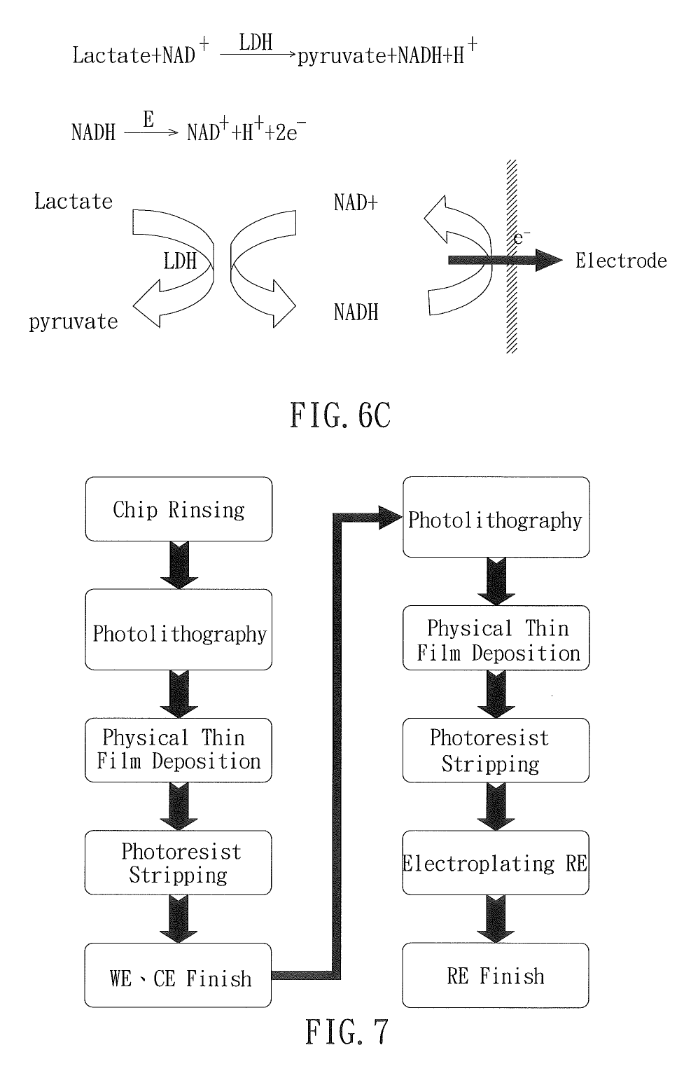

With reference to FIGS. 6A, 6B and 6C for a sensing mechanism of an implantable biosensor system chip biosensor in a wireless monitoring bio-diagnosis system of the present invention, an electrochemical detection electrode of a biosensor in the implantable biosensor system chip of the invention breaks through the conventional electrochemical detection that only can use an oxidation-reduction process of the enzyme for the detection, but the invention uses a charge producing oxidase catalytic reaction (CPOCR) to combine a testing substance such as glucose oxidase with bio molecules and fix the testing substance on a thin film and a substrate. In the meantime, a polarized potential is applied, and glucose oxidase is provided for catalyzing the oxidation of glucose and then producing electrons discharged from the deoxidative decomposition of hydrogen peroxide. The quantity of discharged electrons is used for calculating the protein concentration of the testing substance. Alternately, a substrate stress inducing affinity interaction (SSIAS) can be used, wherein an affinity bond between an antibody and an antigen makes use of the difference of structural stresses produced on a cantilever system to calculate the protein concentration of the testing substance according to a displacement of the cantilever system. In FIG. 6A, a sulfur containing long chain with an end composed of amino groups is formed on a surface of a sensing electrode made of gold, platinum, silver or any other metal, and electrically conductive organic coupling molecules 72 are coupled to a sulfur containing long chain having an end composed of amino groups. After a structure with an end composed of carboxyl groups is formed on a surface, and the testing blood, and the blood adheres antibodies such as C-reactive protein and S-100 protein having an end composed of amino groups on a surface of a sensing electrode to form an adhesive layer, and then adheres various different antigens corresponding to the antibodies such as C-reactive protein and S-100 protein at a surface of the adhesive layer. A change of the electrochemical property on the electrode caused by the adherence of different concentrations can be used for monitoring a change of different physiological parameters effectively. In a detection of a change of sugar concentration and enzyme concentration as shown in FIGS. 6B and 6C, the principle of detecting glucose and lactate sensor adopts glucose oxidase and lactate dehydrogenase (LDH) detections, whose mechanisms are shown in the reaction formulae of FIGS. 6B and 6C. If the potential is too high and the reaction speed is too slow, a catalyst or a mediator such as the potassium ferrocyanide (K₃Fe(CN)₆) in the reaction formula can be used for reducing the detection potential. In the addition of a mediator for reducing the detection potential, a product for changing the reaction formula of an enzyme is used, and an electrochemical method is used for detecting a current change of oxidation and reduction in order to detect the glucose and lactic acid concentration indirectly. Then, an electrochemical instrument is used for adhering the glucose oxidase (GOD) and lactate dehydrogenase (LDH) onto a working electrode by a CV method.

FIG. 7 illustrates a method of manufacturing an implantable biosensor system chip in a wireless monitoring bio-diagnosis system of the present invention, the implantable biosensor system chip for detecting glucose in blood is used for example, and glucose oxidase is mainly used for detecting an electrode voltage of glucose on an electrode, wherein the electrode is made of chromium (Cr), gold (Au), silver (Ag), platinum (Pt) or their alloys, and a chromium film is provided for assisting an attachment of gold (Au) or silver (Ag) films. An electrochemical sensing electrode is manufactured by an electrode manufacturing process as shown in FIG. 7, wherein the procedure of manufacturing the electrode comprises the steps of cleaning a glass substrate; using a mask to produce a required pattern by photolithography; coating a thin film on the glass substrate by thin film deposition; and finally developing the pattern by photoresist stripping, so as to complete manufacturing a working electrode and an auxiliary electrode required in the electrochemical sensing electrode. In addition, the aforementioned procedure is repeated. Similarly, a mask and photolithography are used for producing another geometric pattern, and a physical thin film deposition and a photoresist stripping are used for completing the manufacture of a reference electrode. An implantable biosensor system chip with another type of enzyme or protein can be manufactured by the same procedure by simply replacing glucose oxidase by an enzyme corresponding to the testing substance.

While the invention has been described by means of specific embodiments, numerous modifications and variations could be made thereto by those skilled in the art without departing from the scope and spirit of the invention set forth in the claims.

What is claimed is:

1. A wireless monitoring bio-diagnosis system, comprising:
one or more implantable biosensor system chips;
a surface transmitter; and
an external monitor center;
wherein the implantable biosensor system chip is connected to the surface
transmitter via a wireless network and connected to the external monitor
center via an external network.

2. The wireless monitoring bio-diagnosis system of claim 1, further comprising:
a biocompatible package;
a biosensor; and
a wireless transmitter;
wherein the biosensor and the wireless transmitter are wrapped inside the
biocompatible package.

3. The wireless monitoring bio-diagnosis system of claim 2, wherein the biocompatible package is made of polyurethane, polyethylene, polymethylmethacrylate, polyester, poly tetra fluoro ethylene, polydimethylsiloxane, poly tetramethylene succinate, polymethylmethacrylate or a polymer with a good biocompatibility.

4. The wireless monitoring bio-diagnosis system of claim 2, wherein the biosensor comprises a dielectrophoreis electrode and an electrochemical detection electrode for driving a fluid and separating blood.

5. The wireless monitoring bio-diagnosis system of claim 2, wherein the biosensor comprises an inlet and an outlet for circulating blood.

6. The wireless monitoring bio-diagnosis system of claim 2, wherein the wireless transmitter comprises a radio frequency (RF) power device.

7. The wireless monitoring bio-diagnosis system of claim 1, wherein the implantable biosensor system chip monitors a heartbeat, a blood sugar, an enzyme concentration, a protein concentration or any other physiological signal.

8. The wireless monitoring bio-diagnosis system of claim 7, wherein the implantable biosensor system chip monitors an enzyme or a protein indicating a cardiovascular disease.

9. The wireless monitoring bio-diagnosis system of claim 8, wherein the enzyme is lactate dehydrogenase or glucose oxidase.

10. The wireless monitoring bio-diagnosis system of claim 8, wherein the protein is C-reactive protein or S-100 protein.

11. A manufacturing method of an implantable biosensor system chip of claim 2, comprising the steps of:
cleaning a glass substrate;
coating a thin film on the glass substrate by thin film deposition;
using a mask to produce a pattern by photolithography; and
developing the pattern by photoresist stripping.
